(19) United States
(12) Patent Application Publication
(10) Pub. No.: US 2014/0234888 A1
(43) Pub. Date: Aug. 21, 2014

(54) Method for Improved Selection of Therapeutic Agents

(71) Applicant: Oleksandr Obodnikov, Kiev (UA)

(72) Inventor: Oleksandr Obodnikov, Kiev (UA)

(21) Appl. No.: 13/771,366

(22) Filed: Feb. 20, 2013

(51) Int. Cl. G01N 33/50

(52) U.S. Cl. CPC G01N 33/5008
USPC 435/29

(57) ABSTRACT

The current invention comprises a method of selection of therapeutic agents for treatment of a particular disease in humans or other mammals in which the ratio of the concentrations of thiol (—SH) and disulfide (—SS—) groups is determined in the blood. The therapeutic agent to be tested may be used to treat a neoplastic, infectious, immunological, inflammatory, or other disorder that involves (but is not limited to) the following physiological systems: respiratory, cardiovascular, genitourinary, gastrointestinal (including the liver and pancreas), neurological, musculoskeletal, immunological, and skin.

### RELATED APPLICATIONS

This application claims benefit of provisional application No. 61/633,978, FILED ON Feb. 22, 2012.

### SUMMARY OF THE INVENTION

The current invention comprises a method of selection of therapeutic agents for treatment of a particular disease in humans or other mammals in which the ratio of the concentrations of thiol (—SH) and disulfide (—SS—) groups is determined in the blood. The therapeutic agent to be tested may be used to treat a neoplastic, infectious, immunological, inflammatory, or other disorder that involves (but is not limited to) the following physiological systems: respiratory, cardiovascular, genitourinary, gastrointestinal (including the liver and pancreas), neurological, musculoskeletal, immunological, and skin.

### BACKGROUND OF THE INVENTION

It is well known that any adaptive or pathological process occurs on a background of reactive oxygen species antioxidant system (AOS) and increased free radical biosubstrates. In response, it is activated AOS of the cells. AOS presented low-molecular compounds—radical traps, which include vitamins A, C, E and K, bioflavonoids, low molecular weight thiols (glutathione and ergotionein) and antiperoxide enzymes (superoxide-dismutase, glutathione-peroxidase, glutathione-reductase, catalase, etc.). The end result of such process is adaptation of the organism to new environmental conditions or the failure of adaptive mechanisms, the development of a pathological condition, is determined as a result interrelationship of prooxidant and antioxidant mechanisms, the ability of AOS to protect cells from excess free radicals and peroxides. Given that almost all known diseases are accompanied by increased free radical activity and weakening of the AOS, is a very urgent assessment of these systems with a view to early diagnosis and pathogenesis based treatment of diseases.

### Thiol-Disulfide Status as an Integral Indicator of Adaptation and Nonspecific Resistance.

Years of research carried out under the supervision of Professor Vladimir Sokolovsky (St. Petersburg, Russia) suggest the leading role in the functioning of the adaptive process of AOS low-molecular and macromolecular (proteins) thiol compounds [1-4]. Tiol compounds having in its structure HS-group, it is very well represented in the cell in the form of the tripeptide glutathione and the many proteins and enzymes. These compounds are present in the cell in two states—the reduced (—HS) and oxidized (—SS—), and the concentration of HS-groups of 2-4 times the concentration of SS-groups, as most thiol protein has a physiological activity in the reduced state, and is the primary component of glutathione redox buffer system cells that support a recovery in its environment (Kulinsky V N, Kolesnichenko L S, 1990).

Thiol—proteins are involved in virtually all key biochemical processes in the body: in energy metabolism, ion exchange, conducting nerve impulses to muscle contraction, secretion in, the reception, etc. The role of thiol compounds in cell activity is extremely important and attracts the attention of researchers for a long time. Even in 1936, Selye observed decrease in glutathione levels in the blood of animals in response to the administration of ACTH and suggested the use of this index as a test of stress influence. In the 60s of last century was carried out a large number of clinical studies have shown that a variety of diseases cause the same response—reducing the concentration of HS-groups in the serum of patients, the degree of reduction of the concentration of these groups depended on the severity of the disease: the severe clinical disease is expressed, the lower the level of HS-groups in the serum. Similar changes in the character identified in the study of the influence of various factors on the organism of animals and humans (cold, emotional stress, magnetic field, exercise, etc.). In 1976, Kvakina E B invited to consider the concentration of HS-groups as an indicator of adaptive capacity of the organism, but this hypothesis does not explain the mechanism of quantitative changes of the thiol groups. In 1979, Vladimir Sokolovsky has suggested that, because thiols exist in the cell in two forms—the reduced and oxidized, they represent a single thiol-disulfide system (TDS), in non-specific adaptive response leading importance of these forms (2-HS⇄SS-+2H.).

Numerous clinical and experimental studies have shown that many diseases such as coronary heart disease and myocardial infarction [1], asthma [2], chronic gastro and duodenal ulcer (Pereslegina N A et al, 1993), late toxemia of pregnancy [2], sore throat, diphtheria, infectious mononucleosis, typhoid fever, viral hepatitis, head trauma [1], as well as adverse environmental factors (laser radiation, magnetic field, physical activity, toxins, allergens, noise) indeed lead to a simultaneous change in the level of reduced and oxidized thiol groups. At the same time revealed the following patterns:
thiol—disulfide system (such as protein and low molecular weight of its components) respond to any exposure to internal or external nature of the change in its redox state, which can be characterized by the ratio of the concentration of HS- and —SS-groups (HS/SS), or thiol—disulfide ratio (TDR).
changes in the redox equilibrium in this system are multidirectional (phase) and depend on the strength and duration of the operative factor. The initial change in the thiol-disulfide system, characterized by higher TDR or shift the redox equilibrium toward recovery, followed by changes in the opposite direction—TDR reduction or shift the redox equilibrium toward oxidation, which can be viewed, respectively, as a sign of its activation and/or possible depletion, reflecting the dynamics of the adaptive process. This is confirmed by the following facts: the nature of changes in TDR during prolonged emotional stress repeats called “voltage curve adaptation” by Selye as well as the mirror image of fluctuations in the intensity of free radical oxidation in different phases of adaptation (Arapetyants M G et al, 1988).
the greater source TDR value (i.e. greater buffer capacity of this system) corresponds to and more high level of resistance. Thus, the effect of the noise of the same intensity (90 dB) and duration (30 days) resulted in a multi-directional effects at different times of the year: to reduce the activity of antioxidant enzymes in spring, when the initial level of HS-groups was low, and to the activation of these enzymes in the autumn at high initial concentration of HS-groups (Rodionova L P, Goncharova L L, Makarova I N, 1986). Shleykin A G et al [3] as an example of survival of rats after injection of sodium fluoride has been demonstrated that in the morning hours when blood TDR is 27% higher than in the evening, the life expectancy of rats was 186 min., and in the evening was 78 min.
TDR can be an integral indicator of adaptable opportunities of an organism or a measure of its non-specific resistance, thus it is a more sensitive test of early stress and changes and more accurately reflects the dynamics of the adaptation process than other tests (leukocyte ratio proposed by Garkavy L C et al., 1990), acetylcholinesterase activity [5]). Change the value of TDR shows hidden changes in the conformation of protein molecules (including non-thiol groups) in the early stages of stress. Most of the adequacy of the TDR as a measure of adaptive ability of the organism compared with enzymatic tests was confirmed by the example of the impact of a blue laser (0.44 mcm) in an experiment on rabbits [6].

Booming in recent years, understanding of the molecular mechanisms of adaptation and consistent with the concept of the pathogenesis of Sokolovsky V V on the leading role of redox states of thio-disulfide system in these processes. At present, more and more researchers have come to the conclusion: the primary target of reactive oxygen species are proteins and their oxidative modification leads to changes in the structure of protein molecules and, therefore, their physicochemical and biological properties. Modern researchers consider oxidative damage to proteins as “an early indicator of tissue damage in various pathological conditions”—atherosclerosis, inflammation, neurological disorders, etc. [7].

In light of this, becomes relevant search methods to assess oxidative protein modification, or assessment of the conformational structure of the protein, since it is clear that the enzyme activity by itself does not give of objective information about its damage by free radicals and peroxides, at least in the initial period of stress, and evaluation of the thermodynamic parameters of the enzyme are procedure quite laborious. Some authors [8] proposed a colorimetric method for estimating the oxidative modification of proteins by the formation of colored complexes between oxidized carbonyl amino acid residues with 2,4-dinitrophenylhydrazine. However, as already stated, the change in the TDR objectively reflects the conformational rearrangements of the protein.

Previously [9] for coronary heart disease was also found a direct relationship between the magnitude of blood and strength of TDR lipoprotein complexes of blood plasma and erythrocyte membranes: the lower the TDR value, the more pronounced and the destabilization of the complexes, the degree of reduction of TDR and the stability of lipoprotein complexes depended directly the severity of coronary artery disease and was maximal after myocardial infarction, which is also a confirmation of the value of TDR as an indicator of structural changes of the protein and its complexes.

Revealed relationship between the thiol-disulfide balance and the ability of blood plasma proteins to bind amines (n-fenildiamine) in patients with allergic dermatoses in the acute phase and during remission [3]. According to the authors, the flare was accompanied by reduction and remission—by increase of binding of amines and simultaneously increase the TDR; oxidative modification of proteins is accompanied by the release of free amines (histamine) that the modern view is to trigger allergic conditions. These authors examine these facts as “direct evidence of the influence of thiol—disulfide balance on the conformational state and the binding capacity of blood plasma proteins in relation to amines.” But it is quite true converse: TDR is an indicator of conformational state and the binding capacity of blood plasma proteins in relation to amines.

Thus, the above facts indicate that the TDR reflects the general molecular mechanism of cell damage—oxidative modification of protein and changes in its structure and functions. Obviously, the value of TDR may serve as an objective indicator of early stress and pathological changes.

In medical practice is important as the fact that TDR is required to determine only 2 biochemical analysis: determination of the contents of HS- and SS-groups in the blood or tissues, with the further calculation of the ratio [10]. The main disadvantages of this method of determination of thiol—disulfide status, in our opinion, is the wide variation in its performance under various laboratory methods for determining (amperometric titration, spectrophotometry, chromatography), and the lack of criteria developed by the correlation between the degree of change in the TDR and the severity of a pathological condition in human. Us (Obodnikov O O et al., 2004) proposed a modification of the method for determining the level of adaptation and nonspecific reactivity (LANSR), in which the monitoring of indicators of TDR in whole blood and in cell concentrate blood in vitro for 24 hours followed by cultivation plotting TDR from time to time of cultivation, and the integral index LANSR determined by the ratio of the area under the curve “TDR—during cultivation” in the whole blood to a similar area under the curve of blood cell concentrate (dynamic TDR-DTDR). Proposed laboratory indicator DTDR is a highly informative integral test that does not depend on the laboratory method of its determination and may be used to assess the severity of the objectification of the patient and control dynamic of the disease [11].

### Thiol-Disulfide Ratio in Blood In Vitro as a Criterion for the Selection of Individualized Treatment.

However, we must mention another aspect of the application rate of TDR in current clinical practice, namely, as a laboratory test in vitro to select individual drug therapy, which is especially important for those areas of clinical medicine, where the effectiveness of modern medical facilities remains inadequate. One of such areas is the clinical immunotherapy. It is well known that the appointment of immunoactive drugs in medical practice are based on a comparison with known immunogram indications for specific drug or for short-term results of its effects on some immunological parameters in vitro (the number and/or functional activity of T-lymphocytes, monocytes by NST—test, etc.). However, these approaches cannot adequately assess and predict the effect on integral immune reactivity, metabolic status of the individual patient. Therefore, several researchers have attempted to use TDS as a test system for individual choice of immunoactive drugs and their therapeutic doses in patients with various diseases on the background of secondary immunodeficiency.

One of the first authors who proposed an original method for screening an individual drug use on the basis of TDR—test [12], is Volchek I V (St. Petersburg, Russia). His researched since the late 90s of last century were devoted to the individualization of the choice of immunotherapy in patients with chronic viral hepatitis B and/or C [13-16]. For example, in [13] provides an overview of clinical trials of individualized therapy for chronic hepatitis C (CHC) in two treatment centers in St. Petersburg with the use of screening methods author of drugs for individual patients. This method is based on a study of the influence of drugs on whole heparinized blood in vitro with subsequent determination of cell fraction thiol-disulfide ratio. Method of characteristics should be noted a 1-hour incubation interval of the drug with red blood cells. The test results for each patient was administered a specific drug dose, which increases the maximum SH—/—SS— ratio. It is established that the rate of virologic response to individual therapy for 1 month was 2.5-3 times higher than in the standard therapy with the same medications (<<Reaferon>>, <<Ukraine>>). Prognostic significance of the method of screening drugs for the treatment of patients with CHC was 89.8%. The frequency of complete remission after 6 and 12 months of the individual drugs alone russian interferon (<<Interal>>, <<Reaferon>>) amounted to 75.9% and 62%, respectively. Only 16.7% of patients with CHC viral genotype 1b were sensitive in vitro to <<Reaferon>>, while the proportion of patients who are sensitive to <<Neovir>>, <<Roncoleukin>> and <<Ukraine>>, was 4-5 times higher. In patients with other viral genotypes of CHC such differences were not observed. The authors conclude that the individual treatment of patients with CHC advisable to test in different dosages and use at least two preparations of interferon (IFN). Such individualized therapy in CHC patients allows three or more times more effective treatment of patients with 6-fold lower incidence of side effects, and ten times cheaper treatment by monotherapy with relatively inexpensive russian drugs interferon, used, usually in lower doses than with the standard of therapy.

In [17] considers the peculiarities of individual immunotherapy for genital chlamydia (GC). The results of a comparative evaluation of antibiotic treatment of GC in combination with individually selected immunotherapy suggest that holding prior to treatment of immune testing of drugs for their impact on the state of the TDR in the cytoplasm of red blood cells may be important for reducing the time for treatment of the patients. TDR—testing provides an opportunity to determine the extent biostimulating or cytotoxic effect of drugs on the immune patient's body and predict the effectiveness of therapy. This approach may have particular importance for the treatment of such forms of chlamydia where the diagnosis is established only on the basis of identifying the parameters of the humoral response of the organism to an existing infection.

In [18] studied the effect of immunomodulators on interferon status and TDR whole blood of patients with human papillomavirus infection (PVI) and genital herpes (GH). Revealed that both methods can be used for individualized immunotherapy of patients with PVI and GH. The advantages of testing for TDR are: the ability to test any drug, including without IFN-inducing activity, simplicity, accessibility and opportunity to explore a much larger number of samples obtained to study the influence of drugs in different therapeutic doses.

In all the above studies used the author's way of TDR—testing by Volchek I V [12]. The disadvantages of this method, in our opinion, are the following:
narrow time interval of incubation of the drug with the blood of the patient and the use of only one point in time (1 hour) for determine the TDR does not show enough potential immuno-biochemical reaction to the drug or non-pharmacological therapeutic agent in a real, meaningful therapeutic periods of time (up to 24-72 hours or more);
use for the study of whole heparinized blood of a patient: Heparin, used as an anticoagulant agent, has a significant immunoactive effect and can therefore significantly alter the investigated immunological effects of the drug or drug-free therapeutic agent;
laboratory testing of immunoactive drugs in a dose corresponding to 1:5000 from a therapeutic does not reflect a fundamental difference in the volume of the blood of people depending on their sex and weight;
calculation of effective doses of drugs taken without regard to their bioavailability for different routes of administration to the patient (enterally, intramuscularly, intravenously);
lack of technological regulations for testing the therapeutic effects of non-drug treatments (radiation therapy, physical therapy, energy therapy information).

We have proposed a method of individual selection of therapeutic agents for a particular patient, which is applicable to select non-drug treatments [19]. According to this method, the cultivation of the components of the patient's blood is carried out within 24-72 hours, TDR determined at intervals of 30-120 minutes, build a graph of TDR as a function of time, and the selection of the optimal medical facility means by comparing the areas under the curves of the dependence of TDR and time of cultivation; cultivation whole citrated blood (patient's blood mixed with anticoagulant solution—sodium citrate), and the selection of drugs for the cultivation of blood a patient uses the drug bioavailability in different ways of administration (enteral, intramuscular, intravenous), as well as gender and weight of the patient; in addition, when used as therapeutic agents and anti-infective antineoplastic chemotherapy drugs, radiation therapy, and other anti-tumor therapeutic agents aimed at a direct violation of the viability of the infectious agent or tumor cells, the drug is selected with the smallest area under the graph of the dependence of the TDR—time of cultivation, and when used as therapeutic agents for drugs or drug-free therapeutic effects of immune-modulating, tonic or corrective action information and choose the drug with the largest area under the graph depending on TDR—the time of cultivation.

Thus, it is proposed to carry out a dynamic test of TDR in the interaction of therapeutic agents with the patient's whole blood in vitro. Selection of optimal treatments for their individual therapeutic doses and combinations proposed to control the TDR dynamics within 24-72 hours, with time intervals, taking into account the three main stages of the induced signal transduction and protein synthesis (eg, 30-60 minutes, 2-3 hours, 24-72 hours after the start of testing), thus achieving the maximum opportunity to properly assess the impact of a remedy for such a dynamic figure, what is the TDR.

In [20] considered the possibility of using the TDS-test for the choice of immunotherapy in patients with eczema brushes associated with atopic dermatitis. Aim of this study was to compare the clinical and immunological effectiveness of traditional treatment (topical steroids, antihistamines, vitamins) and the similar treatment in combination with individualized immunotherapy. Selection was carried out among five rival immunodrugs Timalin, Laferon, Pproteflazid, Cycloferon, Amyksin) by in vitro test in the 24-h incubation of the patient's blood with drugs and the subsequent dynamic (before incubation and after 30 minutes, 1 hour, 3 hours and 24 hours after the start of incubation) of the definition of TDS under way [19]. The treatment was used only in those immunodrugs and in single therapeutic doses, which led to maximize the area under the curve “TDR—incubation time” in the TDR—test compared with the control (blood incubated with saline solution). Selected by this method and then carried out in patients with individualized immunotherapy was accompanied by a much more pronounced normalization of the T-cell immunity and positive clinical dynamics of the disease, and at distant follow-up (12 months)—a significant lengthening of the period of remission compared with patients of the control group, receiving only traditional treatment. It is concluded that the additional (to the basic therapy) individualized (based on pre-selection of immunodrugs in TDR—test) immunocorrective therapy promotes a significant increase in the immediate and long-term efficacy of treatment of patients with eczema brushes associated with atopic dermatitis.

A comparative study [21] the effectiveness of standard and individualized immunotherapy in patients with chronic bronchitis (CB). It is established that the pre-immune drug selection among a number of competing on the basis of TDR test of blood in vitro leads to an increase in clinical effect of treatment by 1.4 times (94% vs. 65% of patients, p<0.05), as well as to reduce the number of poor immunological outcomes at 2.4 times (17% vs. 41%, p<0.05).

The method of TDR—testing was also used to select of endocrinotherapy (ET) in patients with genital endometriosis (GE) [22]. There was comparison of clinical efficacy of ET in patients with GE between the traditional selection of endocrine drugs on the basis of clinical and traditional laboratory methods of examination (“hormonal profile”, ultrasound, etc.) or individual choice of ET based on the preliminary assessment of the impact of competing drugs on the TDR level in vitro. Clinical efficacy of individualized ET occurred in 39 (85%) patients, and in the group with the traditional choice of drugs—in 17 (42%) patients. The authors conclude that patients with GE individual choice of endocrine drugs advisable to carry out on the basis of individual sensitivity to the blood of patients in the TDR—test, which enables significantly (2-fold) increase the effectiveness of treatment compared with the conventional approach.

A number of studies [23-25] was carried out to study the efficiency of individual choice of anticancer chemicals (AC) in patients with inoperable form of lung cancer (LC). Prior to the chemotherapy (CT) was determined the individual reaction of the blood in a TDR-test in vitro to a number of competing AC in aliquots corresponding to different therapeutic doses (single and/or course within the therapeutic range). Patients of the main group received individualized chemotherapy, which included only those AC (from 2 to 4), which was revealed sensitivity in TDR-test. Patients of the control group received standard chemotherapy based on conventional protocol, depending on the morphological characteristics of the LC. In patients with advanced non-small cell lung cancer (NSCLC) objective response to treatment was observed in 2 times more likely in individualized CT group (37% vs. 19%, p<0.05), than in the control group; in patients with small cell lung cancer (SCLC) direct objective response rate of individualized CT exceeded the standard by 1.6 times (95.5% vs. 60% of patients, p<0.05). Long-term results after repeated courses of CT (including, in combination with radiotherapy in the standard mode in the absence of contraindications) showed in 2.6 times increase in median survival in individualized CT patients group with NSCLC (24.2 months vs. 9.3 months, respectively, p<0.05) compared with a comparable group of NSCLC patients treated empirically chosen standard regimens of CT.

We investigated the possibility of individual choice of antibacterial drugs in patients with acute exacerbation of chronic bronchitis [26]. There was revealed that in addition to the standard antibiotic therapy, the proposed TDR-test can be used for an adequate choice of effective individualized antibiotic therapy, which is especially important in cases of inability to obtain material for bacteriological examination.

Thus, the first experience with the TDR-test for individual choice of drugs (immune, endocrine, antitumor, antibacterial) gives grounds to consider this approach as quite promising and deserves widespread introduction into clinical practice. TDR—test can be used for improve the effectiveness of treatment of patients and simultaneous for economy of the financial expenses for medicinal treatment.

### The Method of Individual Selection of Therapeutic Agents for a Particular Patient:

The invention relates to medicine, namely, to methods of individual selection (screening) medical facilities: how drugs and drug-free treatments for certain patients with various human diseases (inflammation, cancer, immune deficiency, infections, etc.).

Known as the closest in nature to the claimed method of individual selection of drugs (screening) for a particular patient (see RF Patent No 2150700, IPC G 01 7 N 33/15, publ. 10.06.2000, the Bull. No 16) which involves the cultivation of the components of the patient's blood with the test drugs, the analysis of substrates of blood components before and after cultivation, the definition of the relative concentrations of thiol (—SH) and disulfide (—SS—) groups in the cell fraction of blood (thiol-disulfide ratio—TDR) and the choice on the optimal therapeutic agents, and make a whole heparinized patient's blood, the drug being tested in a dose corresponding to 1:5000 of the therapeutic dose, blood cultured with drug during 1 hour, as a result of choosing the drug with the highest TDR for a particular patient.

The disadvantages of this method are as follows:
as shown by our study, the narrow time interval of incubation of the drug from the blood of the patient and the use of only one point in time (1:00 or 2:00) to determine the TDR is not enough time for potential immuno-biochemical reaction to the drug or non-pharmacological therapeutic agent in a real, therapeutically significant for drug therapy intervals (24-72 hours or more);
use for the study of heparinized blood of a patient: Heparin, used as an anticoagulant agent, is both a means of immunoactive, and therefore affects the reactions, resulting in changes directly effect of the drug or drug-free test therapeutic agent;
laboratory testing of immunoactive drugs in a dose corresponding to 1:5000 from a therapeutic does not reflect a fundamental difference in the volume of the blood of people depending on their sex and weight;
calculation of effective doses of drugs taken without regard to their bioavailability for different routes of administration to the patient (intramuscularly, intravenously, enterally), taking part in the immuno-biochemical reactions;
lack of technological regulations for testing the therapeutic effects of non-drug treatments (physiotherapy, energy information therapy, radiation therapy).

The invention is a task of such improvement in the way of individual selection of drugs and non-drug treatments for a particular patient, which by the choice of other time periods of cultivation and changes in estimates of choosing the optimal therapeutic effect, but also by taking into account a number of other factors to improve the accuracy of the process selection the necessary medical facilities, optimizing doses and combinations that will improve the subsequent therapeutic effect, in addition, there is increasing medical capabilities of the process by choosing the non-drug treatments.

To do this in the way of individual selection of therapeutic agents for a particular patient, according to which the cultivation is carried out with the patient's blood components tested by medical means, analysis of substrate components of the blood before and after cultivation of blood components, determine the ratio of the concentrations of thiol (—SH) and disulfide (—SS —) groups in the blood (thiol-disulfide ratio—TDR) and choose, taking into account the latest best remedy, according to the invention, the cultivation of the components of the patient's blood is carried out within 24-72 hours, TDR determined at intervals of 30-120 minutes, build a graph of TDR as function of time, and the choice of the best means by comparing the areas under the graphs, calculated according to TDR and cultivation time, while cultivating the components of the patient's blood is carried out simultaneously with several means, cultured cell fraction was obtained from citrated blood (the patient's blood mixed with anticoagulant solution—sodium citrate), and the selection of therapeutic agents as drugs for the cultivation of the components of the patient's blood using the drug being tested because of its bioavailability in different ways of administration (intramuscularly, intravenously, enterally) and by sex and weight of the patient, and the selection of as drug-free treatment of their dosing is carried out as follows: for treatment of high power (inductothermy, anticancer radiotherapy) at the time of the patient's blood sample is selected at the same time less therapeutic, how many times the volume of the blood sample is less than the amount of tissue included in the irradiation at carrying out the procedure on a person; for ultralow power of healing and mainly information and corrective action (energy-information therapy) for the duration of the blood sample is selected is the same as for the duration of the patient during the therapeutic procedure, in addition, when used as therapeutic agents anti-infective and anti-viral chemotherapy, radiation therapy, anti-tumor and other therapeutic agents aimed at a direct violation of the viability of the infectious agent or tumor cell, select the drug with the lowest area under the plot of TDR—time of cultivation, and when used as therapeutic agents for drugs or drug-free treatment of immunocorrective, restorative or corrective action information and choose the drug with the largest area under the graph depending of TDR—the time of cultivation.

Causal link between the declared set of characteristics and medical results to that achieved in implementing them, is as follows.

Due to the increase of cultivation time of blood components to 24-72 hours and choose another, qualitatively new evaluation criterion of selection of optimal drug or non-drug therapeutic influence being increased reliability of the choice of drugs and non-drug treatments for a particular patient due to the reflection of the integral dynamics of TDR as one of the most important biochemical indicators of nonspecific resistance.

Effective treatment effects on the patient's body lead to increased TDR in blood, but inefficient—on the contrary, to TDR decline. As shown by our experimental and clinical research, the influence of drugs and non-drug therapeutic remedies for TDR in cultured blood fractions varies in time (within minutes or hours) and the most reliable results can be obtained only by studying the dynamic of this process during 24-72 hours determining the value of TDR an interval of at least 30-120 minutes. This is because, firstly, every drug has a unique pharmacodynamic characteristics that determine the unique temporal changes of biochemical reactions with gradually increasing the time constant, and secondly, a number of drugs (antiviral, antimicrobial, antitumor) and radiotherapy (x-ray, γ-, neutron radiation) have a significant immunosuppressive effect, reflected in the reduction of TDR levels. In addition, the dynamics of reactions to medications and non-drug therapeutic remedies imposed on own biorhythms of the body. Thus, the optimization of the treatment of many diseases largely depends on the correct choice etiotropic and pathogenic agents with multi-directional effects on the immune and biochemical status, and, accordingly, on the TDR levels. Therefore, we believe that in addition to testing the effectiveness of individual drugs or non-drug therapeutic remedies, a more rational is the definition of individual selection for the patient at the same time several drugs or combinations of different therapeutic agents, with immunnoactive therapy should largely prevent or compensate for immunosuppression, which can cause the anti-infection or antitumor drugs. An important element should also be consideration of the bioavailability of each drug at different ways of its introduction into the organism (intramuscularly, intravenously, enterally), and calculate the therapeutic dose based on gender and weight of the patient. The calculation of therapeutic doses is carried out using the formula for calculating the volume of blood in the patient: V=71 ml·P (kg) for men and V=66 ml·P (kg) for women (V—volume of blood, P—weight of the patient). Experimentally we have revealled the correspondence of peaks TDR to different drugs, which depends, firstly, on the properties of these drugs and, secondly, from the time of their cultivation. Therefore, we first proposed to take, in fact, it is a dynamic integrated assessment test “in vitro” level of nonspecific resistance to therapeutic agents during interaction with the blood of the patient. Selection of optimal treatments, their doses and combinations proposed to control the TDR dynamics within 24-72 hours, with some time intervals. The choice of temporary interval for determination the TDR dynamic is defined of main stage of the biochemical interaction of medicine (or non-drug therapeutic influence) with blood of the patient in vitro (phase 1—induction of biochemical reactions within the first 10-40 minutes, 2nd—for 1-3 hours, 3rd—within 24-72 hours). Therefore, TDR is expedient to record in intervals of precisely these three main stages of the induced signal transduction and protein synthesis (30-60 minutes, 2-3 hours, 24-72 hours after the start of testing). The most objective indicators of this process were observed during cultivation of the drug with citrated blood (anticoagulant heparin is an active biochemical agent, greatly distorting influence medicinal therapeutic agents on blood in vitro). The claimed method includes the regulation of the technology to test the therapeutic effect of non-drug treatments (inductothermy, anticancer radiotherapy). At the time of the patient's blood sample is selected at the same time less therapeutic, how many times the volume of the blood sample is less than the amount of tissue included in the field of radiation during the procedure on a person; for ultralow power of healing and mainly information and corrective action (energy information-therapy) for the duration of the blood sample is selected is the same as for the duration of the patient during the therapeutic procedure. This method does not require expensive equipment and reagents and can be implemented in any clinical and biochemical laboratory according to standard methods (amperometric titration, reaction with Elman's reagent etc.).

### Examples of Specific Implementation of the Method of Individual Selection of Treatment for a Particular Patient

In what follows, examples 1 and 2 relate to the individual selection of anticancer cytostatics and immunocorrective therapeutic drugs, and Examples 3 and 4—anti-infective drug selection (antibiotics) and their combination with physiotherapy treatment facility (inductothermy).

### EXAMPLE 1

Patient G., 68 years (med. card No. 243 of 13.09.2000) received treatment at the clinic of Thoracic Surgery and Pulmonology Hospital No. 17 (Kiev, Ukraine), Pulmonology Department at the National Medical Academy of Postgraduate Education. Diagnosis: “Ovarian cancer (adenocarcinoma) with metastases in the right lung; IV clinical stage, IV clinical group.” On 18.09.2000, the patient was planned individualized chemotherapy and immunotherapy.

Individual selection of anticancer chemotherapy was based on testing the dynamics of TDR of blood with cytostatics—Cyclophosphamide (in single therapeutic doses 400-600-800 mg/m2), Cisplatin (50-75-100 mg/m2), Carboplatin (150-300-450 mg/m2), Doxorubicin (25-35-45 mg/m2), Epidoxorubicin (50-70-90 mg/m2), Paclitaxel (135-170-200 mg/m2), Etoposide (100-120-150 mg/m2), Mitomycin C (8-10-12 mg/m2).

Individual selection immunocorrective drugs based on testing the dynamics of TDR of blood with Laferon (therapeutic dose of 1 million IU of 2 million IU, 3 million IU), GMDP—glucosomuramyldipeptide (therapeutic dose of 5-7.5-10 mg), Cycloferon (250-500 mg), in which the cultivation was carried out of blood from each patient from these drugs with a range of therapeutic doses, while substrates analyzed whole blood before and after certain time intervals of cultivation. Calculation of doses of drugs were tested in test tubes, was carried out taking into account the bioavailability of drugs (for intravenous introduction of anticancer cytostatics—100%, for intramuscular introduction of Laferon and Cycloferon bioavailability is 90%, for enteral administration of GMDP—10%) and the estimated volume of blood from the patient (66 ml·78 kg body weight=5148 ml). Determination of TDR was performed by forward and reverse amperometric titration after 1 hour, 2 hours and 24 hours after making the aliquot's dose were cultured in blood in vitro. Dynamics of TDR in the cultivated fraction is given in the Table. 1 and FIG. 1 (attached), which shows graphs of TDS as a function of time. We compared the area under the graphs and concluded that the most anticancer cytostatic action in TDR-test in vitro possessed Carboplatin (in dose 450 mg/m2), Cisplatin (in dose 75 mg/m2), Cyclophosphamide (in dose 600 mg/m2), Paclitaxel (in dose 175 mg/m2): cytostatics was expressed in reduction TDR after 24 hours of incubations on 36%, 32%, 30% and 28%, respectively. This stright reflects the suppression redox balance in blood and obliquely—in tumors of the patient. Thereby, optimum choice of individualized chemotherapy for this patient was determined combination Carboplatin 450 mg/m2+Cyclophosphamide 600 mg/m2 for 1^{st} course of the treatment.

The most immunocorrective activity in the early stages of immuno-biochemical interaction has Laferon (especially at a dose of 2 million IU, where TDR has increased by 30% compared to the control for the 1st hour). In the later stages of incubation (24 h) the greatest ability to increase the TDR showed two other drugs—interferon's inducer Cycloferon (especially at a dose of 250 mg, at which TDR has increased by 101% compared to control) and GMDP (especially at a dose of 5 mg, at which TDR has increased by 64%). Higher effect of lower doses of these drugs compared to the big testified, apparently, a significant depletion of the immune system, whose state at the time of testing match “paradoxical phase parabiotic process” (by Vvedensky N E). At the same time a high level of reaction at the stage of full-scale synthesis of inducible proteins (24) with optimal (low) doses clearly testified that this depletion is mostly not metabolic in nature, and nature of the signal refractoriness. Elimination of this state would contribute the drug that can cause a significant increase in TDR in the early stages of the immune response, biochemical response (in this case Laferon at a dose of 2 million IU). A positive factor is the fact that these drugs (Laferon, Cycloferon and GMDP) do not coincide on the receptor specificity, neither the metabolic pathways of cellular response to them, so their effects are considered additive. Thus, the optimal choice for this patient identified a combination of Laferon dose of 2 million IU+Cycloferon a dose of 250 mg+GMDP at a dosage of 5 mg (a standard two-week course).

After a course of treatment with the inclusion of individual selection anticancer cytostatics and immunocorrective drugs, the patient achieved a partial objective tumor regression and complete response of ovarian metastases in the right lung with the resorption of effusion of metastatic pleurisy.

### EXAMPLE 2

Patient B., 68 years (med. card No. 510 of 10.10.2000); diagnosis: “The central cancer (adenocarcinoma) of the upper lobe of the left lung with metastases in the right lung, mediastinum and neck lymph nodes; IV clinical stage, IV clinical group”. 18.10.2000, the patient underwent surgery (palliative resection of the upper lobe of the left lung). Then individualized chemotherapy and immunotherapy was planned.

Individual selection of anticancer chemotherapy was based on testing the dynamics of TDR in blood with cytostatics—Etoposide (in single therapeutic doses 80-100-120 mg/m2), Cisplatin (25-50-75-100 mg/m2), Carboplatin (150-300-450 mg/m2), Gemcitabine (800-1000-1200 mg/m2), Navelbine (25-30-35 mg/m2), Doxorubicin (20-30-40 mg/m2), Epidoxorubicin (50-70-90 mg/m2), Mitomycin C (4-6-8 mg/m2).

We concluded that the most anticancer cytostatic action in TDR-test in vitro possessed Cisplatin (in dose 100 mg/m2), Gemcitabine (in dose 1200 mg/m2), Carboplatin (in dose 350 mg/m2), Paclitaxel (in dose 190 mg/m2), Navelbine (in dose 25 mg/m2), Etoposide (in dose 120 mg/m2): cytostatics was expressed in reduction TDR after 24 hours of incubations on 32%, 30%, 27%, 25%, 20% and 21%, respectively. Thereby, optimum choice of individualized chemotherapy for this patient was determined combination Cisplatin 100 mg/m2+Gemcitabine 1200 mg/m2 for 1st course of the treatment.

Conducted individual selection of immunoactive drugs among five different medications with their doses (which carried out the cultivation of the blood cell fraction from each of the drugs, with the substrates analyzed cell fraction to its cultivation, and after 1 hour, 2 hours and 24 hours of cultivation), whose results shown in Table. 2 and FIG. 2 (attached), which shows graphs of TDR as a function of time. We compared the area under the graphs and come to this conclusion: in the early stages of interaction (after 1 hour of incubation of drugs with the blood cell fraction of patients) caused the greatest increase in TDR by GMDP a dose of 50 mg (increased by 78% compared with the control) and IL-2 (Interleukin-2) at a dose of 500 000 IU—increasing the TDR by 25%. In the later stages of interaction (after 24 h of incubation) the greatest effect of increasing the TDR had IL-2 in a dose of 500 000 IU (increase by 467%), Laferon dose of 2 000000 IU (increase by 287%) and GMDP 10 mg (increase by 228%). Special attention is given the effect of multiple increase of TDR in control samples in the early stages of incubation (1-2 hours) and a sharp drop in this indicator in the late stage (24 hours).

Based on the clinical and immunological parameters and the results of individual tests of immunodrugs in vitro, the patient was scheduled in conjunction with individualized chemotherapy and combination immunotherapy (IL-2, 500 000 IU+GMDP 10 mg), a two-week course. As a result of individualized chemo-immunotherapy, the patient achieved a partial regression of tumor metastases in the right lung and a significant reduction in the size of metastatic mediastinum and neck lymph nodes.

### EXAMPLE 3

Patient F., aged 16 (med. card No. 741 of 21.12.2000), treated as outpatients. Diagnosis: “Chronic rhinitis of allergic origin, chronic bronchitis, allergic component in the acute stage.” In history: positive allergic reactions to penicillin antibiotics, the lack of reaction to tetracycline antibiotics. Due to the low efficiency of conventional antibacterial (Biseptol, Erythromycin) and allergy (Diazolin, Tavegil) therapy in a patient sample of sputum carried sensitivity of microflora to antibiotics, and individual testing of therapeutic factors as the antibiotic Tetracycline Doxycycline, using the apparatus inductothermy “Magniterm” and the connection these factors. The testing was carried out cultivation of the patient's blood in vitro with the antibiotic Doxycycline, after the effect of non-drug treatment facilities (inductothermy) and combinations thereof, performing analysis of substrates of the cell fraction to its cultivation, and after 1 hour, 2 hours and 24 hours of cultivation. Doxycycline is used in doses of 100 mg and 200 mg on admission; when calculating the duration of induktothermy effect was adopted by the duration of the patient's blood sample for 2 seconds, which corresponds to the length of the actual procedure, 25-30 minutes. Test results are summarized in Table. 3 and FIG. 3 (attached), which shows graphs of TDR as a function of time.

When comparing the areas under the graphs shows that the Doxycycline caused a progressive reduction of TDR in samples of the patient's blood, and this reduction was dose-dependent at early time intervals (1 and 2 hours) and almost independent of the dose within 24 hours of incubation. On the contrary, led to a transient inductothermy reduction of TDR in the early stages and its significant rise (79% above the control) after 24 hours. The combined effect inductothermy and Doxycycline 100 mg resulted in a dramatic (threefold) reduction of TDR in the range 1-2 hours and its almost complete recovery to control values after 24 hours. Based on these data, the patient was prescribed antibiotic therapy and inductothermy the following scheme: 1 capsule Doxycycline (100 mg) per os, followed by 1 hour—a session on the projection area inductothermy major bronchi of both lungs (25 minutes) with two opposing fields of vehicles “Magniterm”; therapy sessions were repeated daily for 5 days.

As a result of treatment the patient achieved normalization of body temperature, the reduction amount of eosinophil cells in the blood and increase amount of lymphocyte cells in the blood to normal levels, the disappearance of cough, dyspnea, and excessive secretions from the nose, which can be regarded as signs of complete remission respiratory diseases.

### EXAMPLE 4

Patient M., 47 years old (med. card. No. 644 of 27.11.2000), the treated as outpatients. Diagnosis: “Chronic obstructive bronchitis.” Feel sick about two years. In history: smoking 20 filtered cigarettes a day, several times during exacerbations, self-involved little success, taking sulfonamides and antibiotics in pill form. The patient was carried out on a sample of sputum sensitivity to antibiotics, and then—individual testing of drugs (based on the results antibiotikogramma) and induktotermy impact. The testing was carried out cultivation of the patient's blood in vitro with antibiotic Cefazolin (Cefalosporin-I) in various doses, after the action of non-drug treatments (inductothermy) and combinations thereof; substrates analyzed cell fraction to its cultivation, and after 1 hour, 2 hours and 24 hours of cultivation, building graphs of TDR as a function of time and comparing the area under the graphs. Testing has shown (Table 4 and FIG. 4, attached) that the microflora of the respiratory tract is highly sensitive to the patient Cefazolin antibiotic, which simultaneously causes a progressive decrease during the incubation of blood samples TDR patient. Inductothermy (action on the blood sample within 2 seconds, which corresponds to the therapeutic procedure duration of 25-30 minutes) resulted in transient reduction of TDR in the first 2 hours, and significantly improving it (by 67% compared to control) after 24 hours of incubation the samples.

Based on the results of investigations the patient was assigned to a combination of antibiotic Cefazolin and inductothermy: endobronchial administration of Cefazolin 250 mg and immediately after this session inductothermy the projection of the large bronchi in the anterior-posterior direction with opposing fields of the two vehicles “Magniterm” for 30 minutes. Exchange treatment for 2 weeks (1 treatment per day termo-antibioticotherapy, with 10 procedures) resulted in sustained remission of chronic bronchitis.

### REFERENCES

1. Thiol compounds in the biochemical mechanisms of pathological processes/Ed. Sokolovsky V V. L., 1979. 88.
2. Antioxidants and adaptation/Ed. Sokolovsky V V. L., 1984. 62.
3. Fluctuations in the state of biochemical systems/Ed. Sokolovsky V V. L., 1986. 75.
4. Sokolovsky V V. Thiol antioxidants in the molecular mechanisms of non-specific reaction to an extreme impact//Vopr. med. chemistry. 1988. Number 34 (6). S. 2-11.
5. Sokolovsky V V, Goncharova L L, Pokrovsky L A, Tyrnova E V. Thiol-compounds and erythrocyte acetylcholinesterase in experimental immobilization stress//Intern. med. overviews. 1993. Number 3. S. 194-196.
6. Goncharova L L, Pokrovskaya L A et al. Role of antioxidant mechanisms in the reactions of the organism to the action of low-intensity laser radiation//Radiation. biol. and radioekol. 1994. No. 34. S. 368-374.
7. Dubinina E E, Shugaley I V. Oxidative modification of proteins//Phys. present-day. biol., 1993. No. 113. S. 71-81.
8. Dubinina E E, Burmistrov S O et al. Oxidative modification of proteins of human serum, the method of its determination//Vopr. med. chemistry. 1995. Number 1. S. 24-29.
9. Alyutova T V, Krylova N I. Redox processes in patients with myocardial infarction in the application “Unithiol” with “Ascorbinic acid”//Heart and blood vessels in health and disease. Saransk, 1983. S. 43-46.
10. Sokolovsky V V. Thiol-disulfide ratio of blood as an indicator of the state of nonspecific resistance: a tutorial. St-Petersburg., 1996. 30.
11. Obodnikov O O, Kochanevich E V, Goncharova Y A. O. Way of the estimation of non-specific resistance level: Patent of Ukraine No. 65948A, 2004 p., Russian Federation Patent for the invention No. 2243559, 2004.
12. Volchek I V. The method of screening for drugs: The Russian Federation Patent number 21,507,700. 1999. Bull. Ns 16, 10.06.2000.
13. Volchek I V, Sologub T N. Nesterov N N and others. Personalized therapy for chronic hepatitis C//Terra Medica Nova (Laboratory diagnosis), 2003. Number 1. P.12-14.
14. Volchek I V, Sologub T V, Belozerova L A. The possibility of individual biologically active treatment of viral hepatitis//Terra Medica Nova. 1999, No. 3, p.14-18.
15. Volchek I V, Sologub T V, Belopol′skaya M A. Individual therapy of chronic hepatitis C with drugs interferon and Neovir//Terra Medica Nova. 2000. Number 3. P.6-9.
16. Volchek I V, Sologub T V, Nesterov N N. Ways to optimize the antiviral therapy of chronic hepatitis C//Terra Medica Nova. 2004. Number 2. C.P-11.
17. Esipov A S, Mikhaylichenko V V, Volchek I V. Personalized immunotherapy for genital chlamydia.//Terra Medica Nova (Laboratory diagnosis). 2004. Number 2. P.11-14.
18. Loginova N S, Loginov V V. Selection of immunomodulators based on the definition of interferon status and thiol-disulfide ratio in patients with human papillomavirus infection and genital herpes//Terra Medica Nova (Laboratory diagnosis), 2004. Number 3. P.12-14.
19. Obodnikov O O, Parshikov O V, Orlovsky O O. Way of the individual selecting the medical facilities for concrete patient. Patent of Ukraine No. 41048A, 2001, Patent Russian Federation for invention No. 2189587, 2002.
20. Kalyuzhnaya L D, Bezvershenko E I, Obodnikov A A. Thiol-disulfide ratio in blood in vitro as a test for the choice of immunotherapy in patients with eczema brushes associated with atopic dermatitis. Zbirnik Naukovich Pratz spivrobitnikiv KMAPE im.P.L.Shupika, 2004. Vip.13. Book 1. P. 122-29.
21. Obodnikov O O. Tiol-disulfide ratio in blood in vitro as test for immunotherapy choice in patients with chronic bronchitis//Nova Medicine, 2005. Number 1. P.58-61.
22. Kochanevich E V, Goncharova Y A, Obodnikov A A. Individualization of endocrinotherapy choice for genital endometriosis patients on the basis of thiol-disulfide ratio in blood in vitro. Zbirnik Naukovich Pratz spivrobitnikiv KMAPE im.P.L.Shupika, 2004. Vip.13. Book 1. S. 435-44.
23. Obodnikov A A Individualization of anticancer chemotherapy choice for patients with advanced NSCLC based on thiol-disulfide ratio in blood in vitro. Zbirnik Naukovich Pratz spivrobitnikiv KMAPE im.P.L.Shupika, 2004. Vip.13. Book 2. S. 97-104.
24. Obodnikov O O, Parshikov O V, Orlovskiy O A. Efficacy of individualized chemotherapy in patients with advanced non-small cell lung cancer//Lung Cancer, 2005. Vol. 49, Suppl. 2. S. 260. P.543.
25. Obodnikov O O. Thiol-disulfide ratio (TDR) in blood as a predictive in vitro test for individual chemotherapy choice in patients with small-cell lung cancer (SCLC). 9th Central Europian Lung Cancer Conference. Gdansk, Poland. Sep. 23-24, 2004//Lung Cancer, 2004. Vol. 45, Suppl. 3. S86.
26. Obodnikov O O, Skachkova O V. Tiol-disulfide ratio in blood in vitro as test for antibiotic-therapy choice in patients with chronic bronchitis//Nova Medicine, 2004. Number 1. P.47-49.

### DETAILED DESCRIPTION OF THE INVENTION

The invention relates to medicine, namely, to methods of individual selection (screening) medical facilities: how drugs and drug-free treatments for certain patients with various human diseases (inflammation, cancer, immune deficiency, infections, etc.) and provides improved accuracy of selection required by medical means, optimize their doses and combinations that will improve further the therapeutic effect, as well as to expand medical facilities through the implementation process for its non-drug treatments.

To do this in the way of individual selection of therapeutic agents for a particular patient, according to which the cultivation is carried out with the patient's blood components tested by medical means, analysis of substrate components of the blood before and after cultivation of blood components, determine the ratio of the concentrations of thiol (—SH) and disulfide (—SS —) groups in the cell fraction (thiol-disulfide ratio—TDR) and choose, taking into account the latest best remedy, according to the invention, the cultivation of the components of the patient's blood is carried out within 24-72 hours, and TDR is determined at intervals of 30-120 minutes, build a graph of TDR as a function of time, and the selection of the optimal means by comparing the areas under the graphs, calculated according to TDR and cultivation time, while cultivating the components of the patient's blood is carried out simultaneously with several therapeutic agents, cultured cell fraction was obtained from citrated blood, and the selection of a therapeutic agents for the cultivation of drugs with the patient's blood components using the drug being tested because of its bioavailability in different ways of administration (intramuscularly, intravenously, enterically) and by sex and weight of the patient, and the selection of therapeutic agents as non-drug treatment of their dosing is carried out as follows:
For intense treatment (inductothermy, anticancer radiotherapy): Determine how many times the volume of the blood sample is less than the amount of tissue included in the field of radiation during the procedure in humans and scale up the treatment dose according to this ratio.
For less intense treatment and mainly corrective action: Exposure of the blood sample to the therapeutic agent is for the same as for the duration during the therapeutic procedure and at the same dose.
When used as therapeutic agents and anti-infective antineoplastic chemotherapy drugs, radiation, antineoplastic therapy and other therapeutic agents aimed at a direct violation of the viability of the infectious agent or tumor cell, choose a drug or therapeutic effect with the smallest area under the TDR graph, depending on the time of cultivation of the therapeutic agent with the patient's blood.
When used as therapeutic agents drugs and drug-free treatment of immune-modulating, tonic or corrective action information, choose a drug or therapeutic effect with the largest area under the TDR graph depending on the time of cultivation of the therapeutic agent with the patient's blood.

1. A method of selection of therapeutic agents for treatment of a particular disease in humans or other mammals in which the ratio of the concentrations of thiol (—SH) and disulfide (—SS—) groups (to be referred to as the thiol-disulfide ratio or TDR) is determined in the blood.

2. A method of claim 1, wherein the therapeutic agent to be tested is used to treat a neoplastic disorder.

3. A method of claim 2, wherein the neoplastic disorder to be tested involves one or more of the following physiological systems: cardiovascular, genitourinary, gastrointestinal (including the liver and pancreas), neurological, musculoskeletal, hematological, and skin

4. A method of claim 1, wherein the type and dose of the therapeutic agent chosen to treat a particular disease is based on in vitro measurements of the difference in TDR between blood treated with a particular therapeutic agent and blood that is not treated with the therapeutic agent.

5. A method of claim 4, wherein the patient's blood is tested in vitro within a period ranging from 24 to 72 hours following withdrawal of the blood from the patient.

6. A method of claim 4, wherein the TDR is tested at intervals of 30 to 120 minutes.

7. A method of claim 4, wherein the response of the blood to the therapeutic agent is determined by measuring the area under the graph produced by measuring TDR values over a given time period.

8. A method of claim 4, wherein the blood is derived from citrated blood.

9. A method of claim 4, wherein the dose of the therapeutic agent tested is based on the patient's specific disorder, age, weight, sex, size of tumor, severity of disease, or other relevant parameter.

10. A method of claim 2, wherein the neoplastic disorder is a carcinoma, lymphoma, leukemia, or sarcoma.
